(19) United States
(12) Patent Application Publication
(10) Pub. No.: US 2005/0019906 A1
(43) Pub. Date: Jan. 27, 2005

(54) Single media in vitro support of fertilized embryos to the implantation stage

(76) Inventors: Michael D. Cecchi, Madison, CT (US); Monica Mezezi, Guelph (CA); Klaus Wiemer, Charlotte, NC (US)

Correspondence Address: William W. Jones, 6 Juniper Lane, Madison, CT, 06443, US

(21) Appl. No.: 10/624,701

(22) Filed: Jul. 23, 2003

(51) Int. Cl. C12N 5/02; C12N 5/08

(52) U.S. Cl. 435/366; 435/404

(57) ABSTRACT

A single culture media system is used to provide an optimal environment for the nutritional requirements for culturing of oocytes and embryos from the retrieval stage of oocytes through the implantation stage of embryos. This latter stage can be through the eight cell, or blastocyst, stage of development. The media of this invention will provide the embryos with a balance of ingredients that provide a consistent and stable composition of ingredients for the embryos whereby each embryo may select ingredients at their own pace or at various times as determined required by the embryos. This single media system provides a substantial improvement over current individual and sequential systems in supplying embryos consistent ingredients, consistent pH levels and osmolality through multiple stage of development so as to reduce the likelihood of osmonic shock, shock resulting from ingredient changes, or by changes in media pH. The embryo implantation stage using the media of this invention can be immediately subsequent to fertilation of the retrieved oocytes, or at the four cell embryo growth stage, or at the eight cell embryo growth, or blastocyst, stage of embryo development. Instead of implanting the fertilized embryos, they can be cryogenically preserved for later implantation.

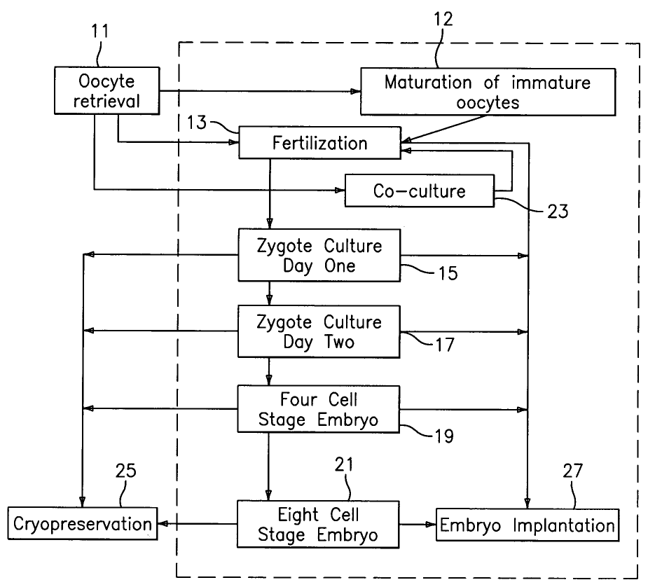

### TECHNICAL FIELD

This invention relates to a single media system and method, and more specifically to a method and media system that uses a single media in human assisted reproduction, which media can supply all of the nutritional requirements for the development of embryos to an implantation stage in vitro. More particularly, the media formulation of this invention will supply necessary nutrients and facilitate development of fertilized oocytes from the fertilization stage to an implantation stage, which implantation stage can be immediately after fertilization of the oocyte, or at the four-cell zygote stage, or at the eight-cell zygote stage. The media of this invention is suitable for culturing the embryos throughout each of the implantation stages, while reducing osmonic shock and stress to the embryos during the culturing process.

### BACKGROUND ART

In vitro culturing of embryos involves the placing of fertilized oocytes which are withdrawn from a donor's ovaries in a culturing dish that contains a media which will support growth of the oocytes, after they are fertilized in the culturing dish. Once fertilized, the embryos will undergo growth by cell multiplication. After about one day the singe cell fertilized embryo will divide into two cells. Further cellular division results in a four cell zygote after about two to three days. After about four to five days the zygote will have grown into an eight cell variant, which is referred to as a blastocyst. Fertilized oocytes, or either the four cell or eight cell variants are deemed to be suitable for implantation back into the donor's womb, or cryopreserved for later implantation.

The culturing media solutions are thus used in the in vitro culturing of embryos and tissues. These media solutions try to mimic body fluids so as to provide the embryos with their nutritional needs to develop and grow. Early culture media used for human assisted reproduction and in vitro fertilization (IVF) were either simple salt solutions such as Earle's balanced salt solution, or a more complex media such as Ham's F-10. These media solutions are either supplemented with maternal or fetal cord serum. Earl's is a simple salt solution composed only of glucose and electrolytes. Ham's F-10 contains essential and non-essential amino adds, vitamins, growth factors, and co-enzymes, along with glucose and electrolytes. These earlier media formulations are basically salt solutions. The problem with these earlier media formulations is that they are too simple, do not provide the embryos with enough of the nutrients that they require, and they can not sustain embryo development into the latter stages of development, such as the blastocyst (eight cell) stage.

Media formulations have also been developed which were thought to more closely approximate fluids such as Human Tubal Fluid (HTF), which was thought to match fetal tubal fluids and provide all the necessary nutrients. These media formulations were thought to be improvements of the aforesaid earlier media, but were found to be unable to sustain blastocyst development.

As the development of media formulations and the needs of embryos became more clearly understood, the ingredients were changed so that the media would become more “stage” specific, and designed for the four different levels of embryo development. Currently, scientists and embryologists use a series of sequential media to provide the nutrients at the various stages of development which sequential media have different formulations. The stages that are addressed by these media are from the retrieval of the oocyte stage to the transfer or implantation of the fertilized embryo back into the uterus stage. There may be three to five different stages, and a different media formulations for each stage of development. Thus, three to five different media formulations are required in the “different media formulation” protocol for embryo culturing.

Currently, there are newer protocols which result in sequentially used different culture media formulations for culturing human embryos. These sequentially used media formulations are designed to enable the culturing of embryos to the blastocyst or eight cell stage. It has been reported that pregnancy rates were doubled when blastocysts are implanted as compared with day three four cell zygotes cultured during the incubation period. These protocols have resulted in an improvement in embryonic development, as shown by the increased number of embryos which attain the blastocyst stage in-vitro. Transfer of blastocysts in turn has led to improved pregnancy and implantation rates of approximately 90% and 60% respectively. A typical clinic may report pregnancy rates following blastocyst transfer of 68% vs. 46% when the four cell embryos were transferred on day three.

Currently most end users and manufacturers use or make a different media formulation for each phase of development. In the area of human reproduction the different phases for individual media products are oocyte retrieval, oocyte maturation, oocyte handling, ordinary fertilization oocyte exam, biopsy zygote exam, and biopsy embryo development to eight cell embryo development to sixteen cell to embryo implantation, as well as the cryopreservation and thawing, the latter of which would encompass a cycle of: oocyte freezing; zygote freezing; embryo freezing; oocyte thawing; zygote thawing; and embryo thawing. Use of the sequential culturing media protocol can require up to eight different culture media formulations.

Proponents of the sequential media protocol claim that the ingredients and components must change between the culturing of cells to eight-cell and the culturing of cells to sixteen cells, and that the requirements change between the zygote stage and the blastocyst stage to account for the significant changes in the physiological and metabolism which occurred during this period. Many of these media changes are documented, such as increases or decreases in pyruvates and glucose, but it is not certain that these media changes occur at the same time that the embryo requires these media changes.

Currently, the manufacturers of media that is used for the blastocyst development stage, embryos from day three to day five, i.e., from the four-cell stage to the eight-cell stage, do not use, and, indeed, make a point of eliminating the use of EDTA in their media. In fact, Gardner Lane, et al. (November 2000) stated: “EDTA should not be used for the later stage embryo as the inhibition of glycolysis reduces energy production at the blastocyst stage and significantly inhibits inner cell mass development”. Vitrolife, Irvine Scientific, and Quinn media formulations (Sage Biopharma) do not include EDTA in the blastocyst stage media.

The absence of EDTA may impact development and add to embryo shock due to changes in the overall media composition and ingredients. Our research does not support these claims and our clinical trials shows no indication that the addition of this material will negatively impact the embryo and its development.

A second problem encountered when using the sequential media protocol is that two or more media formulations are manufactured in different batches and at different times, which may result in their not being consistent and/or compatible, and may not have the same osmolality, or pH. This may cause embryo shock and osmotic shock. This may also result in additional adverse effects on the development of the embryos.

An additional problem with the use of sequential media formulations is that end users may not change the media at the correct time, or they may neglect to use the second media for each particular embryo due to timing or simply forgetting.

Another problem relating to the use of individual or sequential media formulations is that they do not allow the allow the end user to change their plan on what day they expect to transfer the embryo from the culture media to the recipient. In such a case, the end user may originally plan to transfer the embryos a the four cell stage, or at day three of the culturing cycle, and therefore may decide to use a non-sequential media formulation or an individual media formulation. But then, as the development of particular embryos continue, a decision may be made not to transfer the embryos on day three of the culturing cycle. That occurrence might lead to a decision to continue the culturing procedure to day five and to transfer the embryos at day five. This decision would require the use of a new culturing media formulation, which could be an individual media formulation or the second media formulation of a sequential culturing media system. In the case of an individual media formulation, one would need a follow up individual media formulation which would not be compatible with the first media formulation that was used. In the case of a sequential media formulation, one would be using a second media formulation in the sequence that would not be compatible with the first media formulation in the sequence, and which may lack particular essential ingredients which may have been included in the first media formulation in the sequence, and not included in the second media formulation; or ingredients may be in the second media formulation that are intended to work in conjunction with ingredients in the first media formulation. In each such case, subjecting the embryos to a change in media formulation can result in embryonic shock and failed embryo development.

It would be desirable to be able to provide embryos with a variety of ingredients in a pool of available ingredients that they need at various times, so that they could draw a particular ingredient from the pool of ingredients, at various times during embryo development. This would, in effect, provide the embryos with a “menu” of ingredients that they could draw from, when needed, during their growth period.

### DISCLOSURE OF THE INVENTION

This invention relates to an improved method and media system for embryonic development for human reproduction, tissue culture and stem culturing, which is useful in the development of embryos and other cultured materials. The media formulation of this invention is a single culture media formulation for the development of embryos after retrieval from a donor's ovaries. This is single culture media formulation that is used for the development of immature oocytes, after the fertilization stage, development to the four-cell stage, and then development to the eight-cell, or blastocyst stage and as an embryo transfer media formulation. This media formulation is unique in its composition and is a departure from conventional media and current media protocols that are used to culture embryos to these stages.

The present invention involves the use of a single medium for culturing multiple stages of embryo development. The medium formulation of this invention has been defined based on nutritional requirements of oocytes, zygotes and blastocysts, from oocyte retrieval to blastocyst stage to blastocyst implantation. This is a one-system medium for IVF that provides a consistent physiological environment for oocytes through the several stages of embryo development. Comparison studies have been performed comparing the single media system of this invention with the prior art individual and sequential media formulation systems, and have found that the single media system performs equally as well as, or better than, the individual and/or the sequential media systems. The single media formulation used in connection with this invention is suitable for supporting retrieved oocytes from the retrieval stage to a later stage when they are suitable for implantation. The later stages can be: immediately after fertilization; at the four cell embryo development stage; or at the eight cell embryo development stage.

The culture media formulation of this invention includes EDTA for all stages of development from the retrieval of the oocytes through the eight-cell blastocyst development stage of the embryo. This invention, by not requiring the change of types of media, will supply the same consistent osmolality and pH levels, while giving the clinician the opportunity to use the media type, and even the same batch and lot for consistency and/or bottle for each patient and for the embryos being developed. This single cell media formulation is superior to sequential media formulations in that it will allow the embryos to be cultured in a single produced batch for the full one, two, three or five days of development, without moving the embryos at day three to a different medial formulations, which may cause osmotic shock or shifts in pH, or shock created by changes in the media composition.

A major advantage of this invention is the ability to allow the clinician to implant the embryo back into the patient at multiple stages of development while using the same basic media. The transfer may be after day one, two, three, four or day five. The clinician may originally decide to develop embryos to transfer at day three, but if they later decide to not do so, they can continue to culture to day four and still be able to use the same media formulation. The same media formulation can also be used as a transfer media formulation at days three, four, five or six of embryonic culturing and implantation of embryos back into the donor patient.

We have also determined that the media formulation of this invention can be used instead of using a sequential culture media techniques for the development of multiple failure patients. We have determined that the media formulation of this invention will result in a fertilization rate of approximately 50% which is approximately equal to that of a co-culture technique.

A preferred composition of a culturing media formed in accordance with this invention is as follows:

| Ingredient | Concentration (mM) | |
|---|---|---|
| CaC₁₂.2H₂O | 1.7 | |
| D-Glucose | 0.2 | |
| EDTA (diNa) | 0.01 | |
| KCl | 2.5 | |
| KH₂PO₄ | 0.35 | |
| Lactate (Na salt)* | 10.0 | |
| MgSO₄.7H₂0 | 0.2 | |
| NaCl | 95.0 | |
| NaHCO₃ | 25.0 | |
| Sodium Piruvate | 0.2 | |
| Glycine | 0.05 | |
| L-Alanine | 0.05 | |
| L-Arginine HCl | 0.3 | |
| L-Asparagine H₂O | 0.3 | |
| L-Aspartic acid | 0.05 | |
| L-Cystine | 0.05 | |
| L-Glutamic Acid | 0.05 | |
| Alanyl-glutamine | 0.5 | |
| L-Histidine HCl.H₂O | 0.1 | |
| L-Isoleucine | 0.2 | |
| L-Leucine | 0.2 | |
| L-Lysine HCl | 0.2 | |
| L-Methionine | 0.05 | |
| L-Phenylalanine | 0.1 | |
| L-Proline | 0.05 | |
| L-Serine | 0.05 | |
| L-Treonine | 0.2 | |
| L-Tryptophan | 0.025 | |
| L-Tryosine | 0.1 | |
| L-Valine | 0.2 | |
| Gentamycin | 0.01 | μg/ml |
| Phenol Red | 1.5 | μg/ml |

*1.42 ml/L lactate

The maintained pH is between 7.0 and 7.5, with the most desired level being 7.35. The osmolality may range between 260 and 270, with the most desired value being 265.

The concentrations of ingredients are per liter of the media to be produced. The ingredients may be mixed together and then introduced to injection grade ultra pure water.

The following table shows a comparison of the media formulation systems of this invention with a number of earlier media formulation systems which are used for individual or sequential media formulation protocols.

| | Individual | Individual | Sequential | Sequential | Single | Single |
|---|---|---|---|---|---|---|
| Stages of | Media | Media | Media | media | media | media |
| development | Day 3 | Day 5 | Day 3 | Day 5 | Day 3 | Day 5 |
| or process | Transfer | Transfer | Transfer | Transfer | Transfer | Transfer |
| Immature | Media 1 | Media 1 | Media 1 | Media 1 | Media 1 | Media 1 |
| oocytes | | | | | | |
| Fertilization | Media 2 | Media 2 | Media 2 | Media 2 | Media 1 | Media 1 |
| Oocyte Culture | Media 3 | Media 3 | Media 3 | Media 3 | Media 1 | Media 1 |
| Zygote Culture | Media 4 | Media 4 | Media 4 | Media 4 | Media 1 | Media 1 |
| Cleavage Culture | Media 5 | Media 5 | Media 5 | Media 5 | Media 1 | Media 1 |
| (4-cell) | | | | | | |
| Blastocyst Culture | N/A | Media 6 | N/A | Media 6 | N/A | Media 1 |
| (8-cell) | | | | | | |
| Embryo Transfer | Media 6 | Media 7 | Media 7 | Media 7 | Media 1 | Media 1 |
| Number of | 6 | 7 | 6 | 7 | 6 | 7 |
| processes or | | | | | | |
| stages | | | | | | |
| Number of | 6 | 7 | 6 | 7 | 1 | 1 |
| media required | | | | | | |

The above table shows that the individual and sequential media formulations can require the use of six or seven different formulations to culture an oocyte and a resultant embryo from an oocyte retrieval stage to an embryo transfer stage. At the same time, the table demonstrates that the use of a single media formulation formed in accordance with this invention requires only one media formulation for the entire culturing process.

It is therefore an object of this invention to provide a culture media system of one media formlation to be used for the culture and development of embryos to include development of immature oocytes, oocyte fertilization, cleavage development, through the four-cell stage and through to the eight-cell stage, to a transfer or implantation stage or, alternatively to a cryopreservation stage.

It is an additional object of this invention to provide a single media system of the character described that will be more consistent and compatible from the oocyte retrieval stage to the eight-cell embryo growth stage, so as to provide the same menu of ingredients and nutrients at each culturing stage.

It is a further object of this invention to provide a single culturing media system protocol of the character described that will provide embryos with a well balanced physiological and nutritional environment whereby they may select by themselves the nutrients necessary to them through their various developmental stages.

It is another object of this invention to provide a stable environment with consistent pH levels, osmolality levels and basic ingredients, so as not to cause fluctuations in the culturing environment which may cause embryonic shock or complications in embryo development due to these fluctuations.

### BRIEF DESCRIPTION OF THE DRAWING

These and other objects and advantages of the invention will become more readily apparent from the following detailed description of several embodiments of the invention when taken in conjunction with the accompanying drawing, which is a schematic representation that shows the various processes and developmental stages wherein the single menu-type media formulation of this invention is used during the sequence of embryo development from the oocyte retrieval stage to the embryo transfer or cryopreservation stage.

### DETAILED DESCRIPTION OF SPECIFIC EMBODIMENTS OF THE INVENTION

The drawing is a schematic representation showing the processes, procedures and stages of development of embryos with the use of a single culture media formulation of this invention. The oocyte retrieval occurs at stage 11 using a flushing media, which may contain Heparin as an anti coagulant. Immature oocytes will be kept in the culturing media during a maturation period as indicated by stage 12. Mature oocytes are then fertilized in the culture media as shown in stage 13. The retrieved oocytes may be co-cultured with other cells in the media as indicated by stage 23, and then fertilized at stage 13. Upon fertilization, the fertilized embryos may be transferred from the culture medium and implanted back into the recipient as indicated in stage 27, or the fertilized embryos may remain in the culture media for the next developmental stage of embryo or zygote culture as indicated by stage 15, which is the first day of culturing stage. After development of the embryos in culturing stage 15, the embryos may be implanted back to the recipient as indicated by stage 27. Alternatively, the embryos may remain in the single media to the next developmental stage of zygote culture, as indicated by stage 17, which is the second day of culturing stage. After the zygote culturing stage 17, the embryos may be implanted back into the recipient as indicated by stage 27. Alternatively, the embryos may remain in single media to the next developmental stage of four cell stage embryo, which is indicated by stage 19. After this level of culture, i.e., the four cell stage the embryos may be implanted back to the recipient as indicated by stage 27. Alternatively, the embryos may be retained in the single culture media to the eight cell embryo stage 21. After the development to the eight-cell embryo stage, the embryos may then be implanted back to the recipient as indicated by stage 27. It will be appreciated that all of the stages 12-21 of development shown in the drawing involve the use of the single culturing media and do not require separate or different culturing media to be employed to culture the oocytes and embryos.

For multiple failure patients, the physician may choose to use the media of this invention for oocyte co-culturing 23 prior to the fertilization stage and for the day two implantation stage 27. If at retrieval 11, there are immature oocytes present, then the physician may use the media of this invention for the development of immature oocytes 29, and then move the oocytes to the fertilization stage 13 in the media and then through the rest of the process.

The drawing shows that media may be used for oocyte maturation 12, in the fertilization 13, day one zygote culture 15, day two zygote culture 17, four cell embryo culture 19, eight cell zygote stage 21 between oocyte retrieval and zygote implantation 27. It also allows for the physician to transfer the embryos back to the recipient after any of the developmental stages, or maintain the embryos in the media formulation for further culturing. After any of the one day, two day, four cell or eight cell developmental stages, the physician may decide to cryo-preserve the various developmental stages of the embryos as indicated by the the numeral 25.

It will be readily appreciated that the use of a single culturing media from the oocyte retrieval stage to the implantation stage provides the oocytes and embryos with all of the nutrients needed to progress through the various stages of development. The use of a single media for the various stages of development reduces the likelihood of media-induced shock, and also ensures that the quality of the media being used is consistent. The single culturing media formulation of this invention could be used to culture oocytes and embryos in several different ways. One way would be to provide a bath of the single culturing media formulation in a culturing dish and place the retrieved and fertilized oocytes in that dish for the entire culturing time period, up until the embryos are ready to be implanted in the donor's body, which could be anywhere from the two to six day culturing stage. A second way to use the single culturing media formulation of this invention would be to place the fertilized oocytes in a first culturing dish and immerse them in a drop of the single culturing media formulation and culture them for about two days, and then transfer the partially cultured embryos to a second culturing dish which contains the same single culturing media formulation. This transferring procedure could be continued until the embryos are ready for implantation, which can be up to the sixth day of culturing, or when they reach the eight cell stage of development.

Since many changes and variations of the disclosed embodiment of the invention may be made without departing from the inventive concept, it is not intended to limit the invention except as required by the appended claims.

1. A single multi-component embryo culturing media formulation which is operative to promote in vitro culturing of oocytes and fertilized embryos from an oocyte retrieval stage of a culturing process to a fertilized embryo-implanting stage of said culturing process.

2. The culturing media formulation of claim 1 wherein said culturing media formulation is operative to promote culturing of embryos to a four cell stage of development.

3. The culturing media formulation of claim 1 wherein said culturing media formulation is operative to promote culturing of embryos to an eight cell stage of development.

4. The culturing media formulation of claim 1 which includes an EDTA component.

5. The culturing media formulation of claim 1 which includes an alamine glutamine component.

6. The culturing media formulation of claim 1 which includes a gentamyacin component.

7. In an in vitro fertilization process, a procedure for culturing oocytes and fertilized embryos from an oocyte retrieval stage to a fertilized embryo implanting stage, said procedure comprising the steps of:
a) providing a single culturing media formulation;
b) combining said oocytes and said single culturing media formulation;
c) fertilizing said oocytes in said culturing media formulation so as to produce fertilized embryos; and
d) retaining said fertilized embryos in said single culturing media formulation during a time period which is between said retrieval stage and said implanting stage of said culturing procedure.

8. The procedure of claim 7 wherein embryos are grown to a four cell stage of development in said single culturing media formulation at said implanting stage.

9. The procedure of claim 7 wherein embryos are grown to an eight cell stage of development in said single culturing media formulation at said implanting stage.

10. The procedure of claim 7 wherein said culturing media formulation includes an EDTA component.

11. The procedure of claim 7 wherein said culturing media formulation includes an alamine glutamine component.

12. The procedure of claim 7 wherein said culturing media formulation includes a gentamycin component.

13. In an in vitro fertilization process, a procedure for culturing oocytes and fertilized embryos from an oocyte retrieval stage to a fertilized embryo implanting stage, said procedure comprising the steps of:
a) providing a single culturing media formulation;
b) combining said oocytes and said single culturing media formulation;
c) fertilizing said oocytes in said culturing media formulation so as to produce fertilized embryos; and
d) retaining said fertilized embryos in said single culturing media formulation for a time period which is sufficient to produce implantable fertilized embryos that are suitable for implanting back into a donor.

14. The procedure of claim 13 wherein said implantable embryos are four cell stage embryos.

15. The procedure of claim 13 wherein said implantable embryos are eight cell stage embryos.

16. The procedure of claim 13 wherein said implantable embryos are cryo-preserved for subsequent implantation.

17. The procedure of claim 13 including the further step of implanting said implantable embryos back into the donor.

18. The procedure of claim 13 wherein said culturing media formulation includes an EDTA component.

19. The procedure of claim 13 wherein said culturing media formulation includes an alamine glutamine component.

20. The procedure of claim 13 wherein said culturing media formulation includes a gentamycin component.
